(19) United States
(12) Patent Application Publication
(10) Pub. No.: US 2023/0135443 A1
(43) Pub. Date: May 4, 2023

(54) DETERMINING CENTRAL NERVOUS SYSTEM INJURY AND RECOVERY

(71) Applicant: LONDON HEALTH SCIENCES CENTRE RESEARCH INC., London (CA)

(72) Inventor: Douglas FRASER, London (CA)

(73) Assignee: LONDON HEALTH SCIENCES CENTRE RESEARCH INC., London, ON (CA)

(21) Appl. No.: 17/910,700

(22) PCT Filed: Mar. 11, 2021
(86) PCT No.: PCT/CA2021/050331

§ 371 Date: Sep. 9, 2022

Related U.S. Application Data
(60) Provisional application No. 62/988,169, filed on Mar. 11, 2020.

(51) Int. Cl. G01N 33/92; G16H 50/20; G16H 10/40

(52) U.S. Cl. CPC G01N 33/92; G16H 50/20; G16H 10/40

(57) ABSTRACT

A method of diagnosing acquired CNS injury (ACNSI) in a subject comprising: (a) obtaining a test sample from the subject (b) comparing levels of a lipid species or a cohort of multiple lipid species in the test sample with the levels of the single lipid species or the cohort of multiple lipid species in a control sample, or comparing to a normal reference range, (i.e. non-ACNSI (referred to as “normal”) subjects) using quantitative measurements, wherein a change (i.e. a drop or an increase) in the level of the lipid species or the cohort of multiple lipid species in the test sample relative to the control sample is indicative of ACNSI in the subject.

### FIELD OF THE INVENTION

This disclosure relates to the diagnosis, treatment and rehabilitation of central nervous system (CNS) injuries.

### BACKGROUND OF THE INVENTION

Acquired central nervous system (CNS) injury (ACNSI), includes acquired brain injury (ABI) and acquired spinal injury (ASI). ABI and ASI can be traumatic and non-traumatic.

Traumatic brain injury (TBI) is an insult to the brain from an external mechanical force, leading to permanent or temporary impairment of cognitive, physical, and psychosocial functions, with an associated diminished or altered state of consciousness (includes both mechanical and blast injury). The Head Injury Interdisciplinary Special Interest Group of the American Congress of Rehabilitation Medicine defines “mild” TBI as “a traumatically induced physiologic disruption of brain function, as manifested by one of the following: (a) any period of loss of consciousness (LOC); (b) any loss of memory for events immediately before or after the event; (c) any alteration in mental state at the time of the event; and (d) focal neurologic deficits, which may or may not be transient; but where the severity of the injury does not exceed the following: loss of consciousness of approximately 30 minutes or less; after 30 minutes, an initial Glasgow Coma scale of 13-15; and cross post traumatic amnesia no greater than 24 hours period. The Glasgow Coma Scale (GCS) helps defines the severity of a TBI (3-8, severe; 9-12 moderate; 13-15 mild), based on eye, verbal and motor responses. TBI is a major public health concern of epidemic proportions, with an annual incidence of 1.6 to 3.2 million in the United States. According to the Centers for Disease Control and Prevention, National Center for Injury Prevention and Control, mild TBI or mTBI, of which concussion and blast wave injury are subsets, is the most common form, representing nearly 75% of all TBIs. Concussion may be caused by mechanical forces in which the head strikes or is struck by an object, or impulsive forces, in which the head moves without itself being subject to trauma (for example, when the chest hits something and the head snaps forward), or by a pressure wave from a blast. All age groups suffer concussions, from the very young to the elderly. Certain activities are more frequently associated with concussions, including athletics and military service, but they also result from general trauma caused by motor vehicle collisions, falls from height and assaults. Concussions often result in significant acute symptoms and in some individuals, long-term neurological dysfunction.

While diagnosis of moderate to severe TBI is straightforward, mild TBI is under-diagnosed following concussion and explosive events. [“Blast Injuries: Traumatic Brain Injuries from Explosions”, Brainline.org.] That is, while moderate and severe TBI are easily diagnosed based on clinical signs, mild TBI can be missed due to subtle, transient or absent clinical signs. The latter require an objective diagnostic, such as a blood test that is sensitive, specific and reproducible.

Diagnosis of clinically significant mTBI can be difficult, as are the decisions to stop play or activities. It is also unclear when mTBI patients should return to daily activities. Thus, there is great interest in discovery of biomarkers to aid in mTBI, including concussion and primary brain blast injury diagnoses, prognoses, and rehabilitation. At present, no single biomarker has sufficient sensitivity and specificity. Concussions are frequent in sports and can contribute to neurological disability and death. Adolescents are particularly susceptible to concussions, with accurate determination of both the injury and its recovery challenging.

Traumatic spinal cord injuries (TSI; e.g. injuries from spine hyperflexion, hyperextension, lateral stress, rotation, compression, distraction and partial spinal cord transection; often from motor vehicle collisions, falls from height, sports, etc.) and non-traumatic spinal cord injuries (non-TSI; e.g, intervertebral disk disease, interruption of blood supply, infection, electrocution, cancer, radiation, etc.) can also result in mild peripheral symptoms (e.g., an “incomplete” injury). Given the often-subtle nature of TSI and non-TSI injuries, they could be better identified with an objective diagnostic test, such as a blood test, that is sensitive, specific and reproducible.

Non-traumatic brain Injury (non-TBI) and non-TSI include injuries to the brain/spine that are not caused by an external physical force. Non-TBIs and non-TSIs can be the result of an illness, oxygen deprivation, metabolic disorders, aneurysms, cardiac arrest, near-drowning experience, psychological events, stroke, poisoning, infections, inflammation, autoimmune, degenerative, ischemic, metabolic and cancer/radiation-induced brain/spinal injuries, etc. Non-TBI can also result in mildly abnormal neurological symptoms. Given the often-subtle nature of non-TBI injuries, they could be better identified with an objective diagnostic test, such as a blood test, that is sensitive, specific and reproducible.

Concussions remain a major global health care problem (1). Approximately half of all concussions occur in sporting activities (2, 3). Accurate diagnosis of concussion is essential to optimize medical care, to provide timely interventions and to prevent repeat injury prior to healing. Diagnosis of concussion relies on an injury event, which may be a direct blow to the head or as a result of transmitted forces from a blow to the body, as well as standardized clinical testing with concussion tools (4). However, concussion diagnoses are often uncertain as self-reporting of symptoms can be inaccurate (5), and the contribution of other factors, such as chronic pain, can exacerbate symptoms (6).

Adolescents are particularly susceptible to concussions (3) and to their potentially long-lasting neurological effects (7, 8), making accurate diagnoses in this age group critical. To date, neither a single nor cluster of symptoms accurately predict concussions in adolescents, although self-reported headache, head pressure, fatigue, and/or noise and light sensitivity have been identified as useful discriminators of injury (9, 10). Clinical recovery relies solely on resolution of self-reported symptoms and clinical signs, with the recovery period typically longer for adolescents (11-13).

A number of blood protein biomarkers for concussion diagnoses have been investigated, all with marginal success (14). Measured with antibody-based technologies, protein biomarkers increase in the blood of some patients following injury. We recently demonstrated that a wide variety of blood metabolites also change after concussion, and that the injury-induced patterns can be accurately identified with machine learning and advanced analytics (15). Following reduction techniques, the most widely relied upon metabolites for concussion diagnoses were from a single class of metabolite, the glycerophospholipids (GPLs). Phosphatidylcholines (PCs), a specific class of GPLs, make up 20-26% of human brain dry weight (16) and are critical for membrane structure, generation of second messengers and regulation of neuronal apoptosis, transporter activities and membrane-bound enzymes (17).

WO 2016/149808 (WO '808), the contents of which are incorporated herein by reference, demonstrates that accurate diagnosis of ACNSI (acquired CNS injury), which includes acquired brain injury and acquired spinal injury, can be aided by metabolomic profiling and machine learning. WO '808 compares a metabolomics profile of an injured subject to what is believed to be a normal cohort of individuals. Accurate diagnosis was obtained by comparing profiles of at least 17 metabolites, but the direction of metabolite change and the recovery of the subjects was not assessed. The 17 metabolites were measured with two techniques, mass spectrometry (MS) and NMR. They may also need collection devices and different standards for each metabolite.

It would be beneficial to have a small number of diagnostic markers measured with MS or any other applicable technique that could serve not just to obtain an accurate diagnosis of injuries of the central nervous system, such as concussion and blast wave injury, but also that could serve to follow up the recovery of the patients.

### SUMMARY OF THE INVENTION

The present disclosure relates to the measurement of GPLs, including PCs, to aid in the diagnoses, treatment and recovery of central nervous system (CNS) injuries.

In accordance with the present disclosure, a method of diagnosing acquired central nervous system injury (ACNSI) in a subject comprises: (a) obtaining a test sample from the subject, and (b) comparing levels of a single lipid species or a cohort of multiple lipid species in the test sample with the levels of the lipid species or the cohort of multiple lipid species in a control sample using quantitative measurements, wherein a change in the level of the single lipid species or the cohort of multiple lipid species in the test sample relative to the control sample is indicative of ACNSI in the subject.

In one embodiment of the method of diagnosing ACNSI of the present disclosure, the method further comprises obtaining a sample from the subject during the subject's recovery for ACNSI, wherein an increase in the levels of the single lipid species or the cohort of multiple lipid species in the recovery sample relative to the levels obtained in the test sample is indicative of a normalization of the subject.

In another embodiment of the method of diagnosing ACNSI of the present disclosure, the control sample is a pre-existing profile of ACNSI for the single lipid species or for the cohort of multiple lipid species.

In another embodiment of the method of diagnosing ACNSI of the present disclosure, the control sample is a pre-existing profile of non-ACNSI for the single lipid species or for the cohort of multiple lipid species.

In another embodiment of the method of diagnosing ACNSI of the present disclosure, the control sample is obtained from the subject at baseline.

In another embodiment of the method of diagnosing ACNSI of the present disclosure, the ACNSI is mild traumatic brain injury (mTBI), mild traumatic spinal cord injury (mTSI), or stroke, poisoning, chemical, infection, autoimmune, hypoxic, ischemic, metabolic or cancer-induced brain or spinal injuries.

In another embodiment of the method of diagnosing ACNSI of the present disclosure, the ACNSI is concussion or blast wave injury.

In another embodiment of the method of diagnosing ACNSI of the present disclosure, the ACNSI is concussion.

In another embodiment of the method of diagnosing ACNSI of the present disclosure, the ACNSI is concussion, and step (b) comprises comparing the levels of the cohort of multiple lipid species, the cohort of multiple lipid species being PC ae C36:0, PC aa C42:6 and PC ae C36:2.

In another embodiment of the method of diagnosing ACNSI of the present disclosure, the ACNSI is concussion, and step (b) comprises comparing the levels of the cohort of multiple lipid species, the cohort of multiple lipid species being PC ae C36:0, PC aa C42:6, PC ae C36:2 and PC aa C32:0.

In another embodiment of the method of diagnosing ACNSI of the present disclosure, step (b) comprises comparing the levels of the single lipid species, the single lipid species being selected from the lipid species included in Table 1.

In another embodiment of the method of diagnosing ACNSI of the present disclosure, the ACNSI is concussion, and step (b) comprises comparing the levels of the single lipid species, and the single lipid species is PC ae C36:0, PC aa C42:6, PC ae C36:2 or PC aa C32:0.

In another embodiment of the method of diagnosing ACNSI of the present disclosure, the sample is a blood sample, a plasma sample, a serum sample, a capillary sample, a sweat sample, a tear sample, a breath sample or a combination thereof.

In another embodiment of the method of diagnosing ACNSI of the present disclosure, the method further comprises (c) treating the subject for ACNSI when there is a change in the level of the single PC or cohort of multiple PCs in the test sample relative to the control sample.

In another embodiment, the present disclosure is a use of a single lipid species or a cohort of multiple lipid species in the diagnosis and treatment of ACNSI.

In one embodiment of the use of a single lipid species or a cohort of multiple lipid species in the diagnosis and treatment of ACNSI of the present disclosure, the ACNSI is mild traumatic brain injury (mTBI), mild traumatic spinal cord injury (mTSI), or stroke, poisoning, chemical, infection, autoimmune, hypoxic, ischemic, metabolic or cancer-induced brain or spinal injuries.

In another embodiment of the use of a single lipid species or a cohort of multiple lipid species in the diagnosis and treatment of ACNSI of the present disclosure, the ACNSI is concussion or blast wave injury.

In another embodiment of the use of a single lipid species or a cohort of multiple lipid species in the diagnosis and treatment of ACNSI of the present disclosure, the single lipid species or cohort of multiple lipid species are selected from Table 1.

In another embodiment of the use of a single lipid species or a cohort of multiple lipid species in the diagnosis and treatment of ACNSI of the present disclosure, the ACNSI is concussion and the cohort of multiple lipid species include PC ae C36:0, PC aa C42:6 and PC ae C36:2.

In another embodiment of the use of a single lipid species or a cohort of multiple lipid species in the diagnosis and treatment of ACNSI of the present disclosure, the ACNSI is concussion and the cohort of multiple lipid species are PC ae C36:0, PC aa C42:6, PC ae C36:2 and PC aa C32:0.

In another embodiment of the use of a single lipid species or a cohort of multiple lipid species in the diagnosis and treatment of ACNSI of the present disclosure, the ACNSI is concussion and the single lipid species is PC ae C36:0, PC aa C42:6, PC ae C36:2 or PC aa C32:0.

In accordance with the present disclosure, an ACNSI diagnostic apparatus includes a computer readable storage medium and a computer program mechanism embedded therein, the computer program mechanism comprising executable instructions for performing a method of diagnosing ACNSI in a subject, said executable instructions comprising: (a) comparing levels of a single lipid species or a cohort of multiple lipid species in a test sample of the subject obtained after an injury of the CNS, with the levels of the lipid species or the cohort of multiple lipid species in a control sample, and (b) providing an ACNSI positive signal when there is a change in the level of the single lipid species or in the levels of the cohort of multiple lipid species in the test sample relative to the control sample is indicative of ACNSI.

In one embodiment of the ACNSI diagnostic apparatus of the present disclosure, the instructions further include comparing the levels of the single lipid species or of the levels of the cohort of multiple lipid species of the subject, with the levels of the single lipid species or of the cohort of multiple lipid species in a sample obtained from the subject during the subject's treatment of ACNSI, wherein an increase in the level of the single lipid species or in the levels of the cohort of multiple lipid species during the treatment relative to the levels of the lipid species or the cohort of multiple lipid species in the test ample is indicative of a normalization of the subject.

In another embodiment of the ACNSI diagnostic apparatus of the present disclosure, the control sample is a pre-existing profile of ACNSI for the single lipid species or the cohort of multiple lipid species.

In another embodiment of the ACNSI diagnostic apparatus of the present disclosure, the control sample is a pre-existing profile of non-ACNSI for the single lipid species or the cohort of multiple lipid species.

In another embodiment of the ACNSI diagnostic apparatus of the present disclosure, the control sample is obtained from the subject at baseline.

In another embodiment of the ACNSI diagnostic apparatus of the present disclosure, the ACNSI is mild traumatic brain injury (mTBI), mild traumatic spinal cord injury (mTSI), or stroke, poisoning, chemical, infection, autoimmune, hypoxic, ischemic, metabolic or cancer-induced brain or spinal injuries.

In another embodiment of the ACNSI diagnostic apparatus of the present disclosure, the ACNSI is concussion or blast wave injury.

In another embodiment of the ACNSI diagnostic apparatus of the present disclosure, the ACNSI is concussion and the cohort of multiple lipid species are PC ae C36:0, PC aa C42:6 and PC ae C36:2.

In another embodiment of the ACNSI diagnostic apparatus of the present disclosure, the ACNSI is concussion, and wherein the cohort of multiple lipid species are PC ae C36:0, PC aa C42:6, PC ae C36:2 and PC aa C32:0.

In another embodiment of the ACNSI diagnostic apparatus of the present disclosure, the ACNSI is concussion and instruction (a) comprises comparing the levels of the single lipid species, and wherein the single lipid species is PC ae C36:0, PC aa C42:6, PC ae C36:2 or PC aa C32:0.

In another embodiment of the ACNSI diagnostic apparatus of the present disclosure, the single lipid species or cohort of multiple lipid species are selected from Table 1.

In accordance with the present disclosure, a computer program product for use in conjunction with a computer system comprises: a computer readable storage medium and a computer program mechanism embedded therein, the computer program mechanism comprising executable instructions for performing a method of diagnosing acquired central nervous system injury (ACNSI) in a subject, said executable instructions comprising: (a) comparing levels of a single lipid species or a cohort of multiple lipid species in a test sample of the subject obtained after an injury of the CNS, with the levels of the lipid species or the cohort of multiple lipid species in a control sample, and (b) determining if the subject has ACNSI based on said comparison.

In one embodiment of the computer program product for use in conjunction with a computer system of the present disclosure, the control sample is a pre-existing profile of ACNSI for the single lipid species or the cohort of multiple lipid species.

In another embodiment of the computer program product for use in conjunction with a computer system of the present disclosure, the control sample is a pre-existing profile of non-ACNSI for the single lipid species or the cohort of multiple lipid species.

In another embodiment of the computer program product for use in conjunction with a computer system of the present disclosure, the control sample is obtained from the subject at baseline.

In another embodiment of the computer program product for use in conjunction with a computer system of the present disclosure, the ACNSI is mild traumatic brain injury (mTBI), mild traumatic spinal cord injury (mTSI), or stroke, poisoning, chemical, infection, autoimmune, hypoxic, ischemic, metabolic or cancer-induced brain or spinal injuries.

In another embodiment of the computer program product for use in conjunction with a computer system of the present disclosure, the ACNSI is concussion or blast wave injury.

In another embodiment of the computer program product for use in conjunction with a computer system of the present disclosure, the ACNSI is concussion and executable instruction (a) comprises comparing the cohort of multiple lipid species, and wherein the cohort of multiple lipid species are PC ae C36:0, PC aa C42:6 and PC ae C36:2.

In another embodiment of the computer program product for use in conjunction with a computer system of the present disclosure, the ACNSI is concussion and executable instruction (a) comprises comparing the levels of the cohort of multiple lipid species, and wherein the cohort of multiple lipid species are PC ae C36:0, PC aa C42:6, PC ae C36:2 and PC aa C32:0.

In another embodiment of the computer program product for use in conjunction with a computer system of the present disclosure, the ACNSI is concussion and executable instruction (a) comprises comparing the levels of the single lipid species, and wherein the single lipid species is PC ae C36:0, PC aa C42:6, PC ae C36:2 or PC aa C32:0.

In another embodiment of the computer program product for use in conjunction with a computer system of the present disclosure, the single lipid species or the cohort of multiple lipid species are selected from Table 1.

In another embodiment, the present disclosure is a kit for use in the method or the use of or the apparatus or the computer program of any one of the previous embodiments, the kit comprising one or more reagents for the detection of the cohort of multiple lipid species in the test, control samples and samples obtained during treatment, and instructions for use.

In one embodiment of the kit, the instructions for use include instructions to seek treatment of the subject for ACNSI.

### BRIEF DESCRIPTION OF THE DRAWINGS

The following figures illustrate various aspects and preferred and alternative embodiments of this disclosure.

FIG. 1 Plots demonstrating the significant reductions in 3 individual PCs after concussion. The y-axis is plasma concentration in μM. P-values and AUCs on ROC curve analyses are listed for each PC.

FIG. 2A final ROC curve analysis for the combination of 3 PCs of FIG. 1. The AUC, 95% confidence intervals (CIs) and P value are indicated. The diagonal solid line reflects an AUC of 0.5 attributed to chance.

FIG. 3A plot demonstrating the recovery of 3 PCs of injured subjects of FIG. 1 (n=5 patients/point). The time points reflect the plasma concentrations post-injury (<72 hours) and repeat sampling at medical clearance (48-72 days). The data are expressed as % control determined from the non-injured group.

FIG. 4 Plots demonstrating the significant reductions in 4 individual PCs after concussion. The y-axis is plasma concentration in 04. P-values and AUCs on ROC curve analyses are listed for each PC. The dotted horizontal lines on each plot reflect the cut off values.

FIG. 5A final ROC curve analysis for the combination of 4 PCs. The AUC, 95% confidence intervals (CIs) and P value are indicated. The solid line reflects an AUC of 0.5 attributed to chance.

FIG. 6A plot demonstrating the recovery of 4 PCs (n=5 patients/point). The time points reflect the plasma concentrations post-injury (<72 hours) and repeat sampling at medical clearance (48-72 days). The data are expressed as % control determined from the non-injured group.

### DESCRIPTION OF THE INVENTION

Abbreviations

Unless defined otherwise, all technical and scientific terms used herein have the same meaning as commonly understood by one of ordinary skill in the art to which this disclosure belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this disclosure.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

“Baseline” means a measurement of reference of a single or multiple lipid species levels in a subject before an event that produces an acquired central nervous system (CNS) injury in the subject, or prior to the start of an activity, such as prior to a sports season.

In this document the definition of “mild traumatic brain injury”, “mTBI””, which may also be referred to in the literature as mild head injury or concussion, is that taken from the American Congress of Rehabilitation Medicine (ACRM; J Head Trauma Rehabil 1993; 8(3):86-87), and it refers to a person who has had a traumatically induced physiological disruption of brain function, as manifested by at least one of the following: 1) any period of loss of consciousness; 2) any loss of memory for events immediately before or after the event; 3) any alteration in mental state at the time of the event (e.g., feeling dazed, disoriented, or confused); and 4) focal neurological deficit(s) that may or may not be transient; but where the severity of the injury does not exceed the following: loss of consciousness of approximately 30 minutes or less; after 30 minutes, an initial Glasgow Coma Scale (GCS) of 13-15; and posttraumatic amnesia (PTA) not greater than 24 hours. This definition includes: 1) the head being struck, 2) the head striking an object, and 3) the brain undergoing an acceleration/deceleration movement (i.e., whiplash) without direct external trauma to the head. Computed tomography, magnetic resonance imaging, electroencephalogram, near infrared spectroscopy, positive emission tomography or routine neurological evaluations may be normal. Due to the lack of medical emergency, or the realities of certain medical systems, some patients may not have the above factors medically documented in the acute stage. In such cases, it is appropriate to consider symptomatology that, when linked to a traumatic head injury, can suggest the existence of a mTBI.

“Non-traumatic brain injuries” (non-TBI) include brain injuries that may be the result of strokes, poisonings, psychological distresses, chemicals, infections, inflammation, autoimmune diseases, degenerative processes, hypoxia, ischemia, metabolic derangements and cancer/radiation.

In this document the definition of “mild traumatic spinal cord injury” “mTSI” is an incomplete injury with one or more spinal symptoms that may resolve over time (e.g. loss of bowel or bladder control, poor regulation of blood pressure and body temperature, pain, poor sensation, poor sense of body position, sexual dysfunction, etc.). Causes of mTSI may include contusion, stretch and partial cord transection.

“Non-traumatic spinal cord injuries” (non-TSI) include spinal cord injuries that may be the result of strokes, poisonings, chemicals, infections, inflammation, autoimmune diseases, degenerative processes, hypoxia, ischemia, metabolic derangements and cancer/radiation.

“Metabolome” refers to the collection of all metabolites in a biological cell, tissue, organ or organism, which are the end products of cellular processes. Metabolites in the metabolome may be endogenous (produced and metabolized by the individual themselves) or exogenous (obtained from external sources and metabolized within the individual, either by host metabolism or microflora) in origin. Metabolites may be altered by lifestyle factors and the microbiome. “Metabolome” includes lipidome, sugars, nucleotides and amino acids. Lipidome is the complete lipid profile in a biological cell, tissue, organ or organism.

“Metabolomic profiling” refers to the characterization and/or measurement of the small molecule metabolites in biological specimen or sample, including cells, tissue, organs, organisms, or any derivative fraction thereof and fluids such as blood, blood plasma, blood serum, capillary blood, venous blood, saliva, synovial fluid, spinal fluids, urine, bronchoalveolar lavage, tissue extracts, tears, volatile organic compounds (VOCs), breath samples, sweat, and so forth. This characterization may be targeted (limited to a defined number of specific compounds) or untargeted/nontargeted in nature (not limited to a defined or known number of compounds).

The metabolite profile may include information such as the quantity and/or type of small molecules present in the sample. The ordinarily skilled artisan would know that the information which is necessary and/or sufficient will vary depending on the intended use of the “metabolite profile.” For example, the “metabolite profile,” can be determined using a single technique for an intended use but may require the use of several different techniques for another intended use depending on such factors as the disease state involved, the types of small molecules present in a particular targeted cellular compartment, the cellular compartment being assayed per se., and so forth.

The relevant information in a “metabolite profile” may also vary depending on the intended use of the compiled information, e.g., mass spectrum or chromatogram. For example, for some intended uses, the amounts of a particular metabolite or a particular class of metabolite may be relevant, but for other uses the distribution of types of metabolites may be relevant.

Metabolite profiles may be generated by several methods, e.g., liquid chromatography (LC), high performance LC (HPLC), ultra-high performance LC (UHPLC), ultra-performance LC (UPLC), thin layer chromatography (TLC), electrochemical analysis, Mass Spectrometry (MS), tandem Mass Spectrometry (MS/MS), time-of-flight mass spectrometry (TOF-MS), refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), fluorescence spectroscopy, dual polarization interferometry, flame ionization detection (FID), computational methods, =Light Scattering analysis (LS), gas chromatography (GC), or GC coupled with MS, direct injection (DI) coupled with MS/MS and/or other methods or combination of methods known in the art.

The term “subject” as used herein refers all members of the animal kingdom including mammals, preferably humans.

The term “patient” as used herein refers to a subject that is suspected of having an acquired injury of the central nervous system (ACNSI). In this document ACNSI includes an acquired brain injury (ABI) and an acquired spinal cord injury (ASI). These injuries may be traumatic (mTBI and mTSI) and non-traumatic (non-TBI and non-TSI). mTBI includes concussion and blast, including blast overpressure wave injury. Non-TBI includes electrical-induced brain injury (electrocution), seizure-induced brain injury, surgical-induced brain injury, stroke-induced brain injury, poison-induced brain injury, psychological brain injury, chemical brain injury, infectious brain injury, ischemic brain injury, metabolic brain injury, inflammatory brain injury, autoimmune brain injury, degenerative brain injury, hypoxic brain injury, and cancer/radiation-induced brain injury. mTSI includes spinal cord contusion, stretch and/or partial transection, and the non-TSI includes intervertebral disk disease, electrical, stroke, poisoning, chemical, infectious, ischemia, metabolic, inflammatory, autoimmune, degenerative, hypoxic, and cancer/radiation-induced spinal cord injuries.

Overview

The present disclosure relates to the use of at least one lipid species, i.e. a single lipid species or a cohort (group of more than 1) of lipid species, including GPLs such as PCs to accurately diagnose, treat and follow up the recovery of acquired central nervous system injuries (ACNSI), including ABI and ASI. ABI includes mTBI and non-TBI. ASI includes mTSI and non-TSI. In the case of concussions, treatment may include rest and supportive care, followed by gradual return to activities. It is imperative to protect against a repeat concussion, particularly while healing. There are symptom therapies (i.e. drugs for headaches, anxieties and depression, and sleep disturbances, as well as educational and cognitive support) and rehabilitation steps, including neck strengthening, aerobic exercise, massage, etc.).

In one embodiment, the approach of the present disclosure involves obtaining a sample from the subject at baseline (i.e. before the subject suffers a CNS injury), shortly after a suspected CNS injury, and again during the recovery period. The lipid species measurements in the samples taken from the same subject at the different times (baseline, post-injury and recovery) are compared using quantitative or semi-quantitative measurements for the levels of at least one lipid species for changes after injury (baseline vs. after suspected injury), and again during recovery to show normalization of metabolite levels. A change, such as a drop or an increase, in the level of the at least one lipid species in the post-injury sample being indicative of ACNSI. The approach of this embodiment is useful of mTBI and mTSI, including those instigated by mechanical, blast, chemical and psychological trauma. Rather than looking at predefined patterns of metabolites, this embodiment focuses on a change in a single lipid species or in a cohort of lipid species. This embodiment is advantageous for individuals whose baseline blood/plasma, capillary blood samples can be obtained because they are about to take part in an activity with risk of injury, such as sports and military service. It may also be conceivable that insurance companies may require athletes as well as subjects involved in specific activities, such as police, firefighters, construction workers, drivers, to take baseline samples before granting insurance to those subjects.

In another embodiment, the present disclosure involves comparing the levels of a single lipid species or a cohort of lipid species in a subject's sample, such as blood, blood plasma, blood serum, capillary blood, venous blood, saliva, synovial fluid, urine, spinal fluid, bronchoalveolar lavage, sweat, tears, breath samples, VOCs and extracts, using quantitative measurements of said cohort of lipid species to the levels of said single lipid species or cohort of lipid species in a known reference range, or in a normal population. A change, such as a drop/decrease or an increase in the level of the at least one lipid species in the subject's sample being indicative of the subject having ACNSI.

The methods and computer programs of the present disclosure may be used in point-of-care metabolomics testing with portable, table/countertop or handheld instruments that generate metabolite profiles.

Single or Cohort of Lipid Species

In one embodiment, the lipid species and the cohort of lipid species are from the lipid species listed in Table 1.

In one embodiment, the cohort of lipid species, including GPLs, include no more than 50 lipid species, such as 2, 3, 4, 5, 6, 7, 8, 9, 10, 11, 12, 13, 14, 15, 16, 17, 18, 19, 20 and so forth to 50 lipid species. In another embodiment, the cohort of lipid species include no more than 25 lipid species. In another embodiment, the cohort of lipid species include no more than 6 lipid species. In another embodiment, the cohort of lipid species include no more than 5 lipid species. In another embodiment, the cohort of lipid species include no more than 4 lipid species. In another embodiment, the cohort of lipid species include no more than 3 lipid species. In another embodiment, the cohort of lipid species include no more than 2 lipid species. The lipid species include GPLs, including PCs.

In one embodiment of the present disclosure, the cohort of lipid species include PC ae C36:0, PC aa C42:6 and PC ae C36:2.

In another embodiment of the of the present disclosure, the cohort of lipid species are PC ae C36:0, PC aa C42:6, PC ae C36:2 and PC aa C32:0.

In another embodiment of the present disclosure, the lipid species is a single lipid species. The single lipid species is, in one embodiment, selected from the lipid species listed in Table 1. In another embodiment the single lipid species is PC ae C36:0, PC aa C42:6, PC ae C36:2 or PC aa C32:0.

Since metabolites exist in a very broad range of concentrations and exhibit chemical diversity, there is no one instrument that can reliably measure all of the metabolites in the non-human or human metabolome in a single analysis. Instead, practitioners of metabolomic profiling generally use a suite of instruments, most often involving different combinations of liquid chromatography (LC) or gas chromatography (GC) coupled with MS, to obtain broad metabolic coverage [Circulation. 2012; 126: 1110-1120]. Other instruments such as electrochemical analysis, RI, UV, near-IR, LS, GC coupled to non-MS detectors and so forth may also be used.

Point-of-care testing (e.g. table top MS) could be developed to identify ACNSI, including mTBI and non-TBI patients, and to prognosticate their brain injuries.

A library of lipid species measurements may be established for diagnosed ABI cases, including mTBIs and non-TBIs. For example, a library of PC measurements of concussion, primary blast in blast-induced traumatic brain injury, electrical-induced brain injury (electrocution), seizure-induced brain injury, surgical-induced brain injury, stroke-induced brain injury, poison-induced brain injury, psychological brain injury, chemical brain injury, infectious brain injury, ischemic brain injury, metabolic brain injury, inflammatory brain injury, autoimmune brain injury, degenerative brain injury, hypoxic brain injury, and cancer/radiation-induced brain injury and any other possible form of ABI. This library may be used as the predetermined, control set of PC measurements of ABI. Similarly, libraries may be established for diagnosed ASI cases to obtain predetermined set of PC measurements of ASI. A predetermined set of normal lipid species measurements may be obtained from subjects known not to have a form of ABI and/or ASI. A comparison may be made of the subject's lipid species measurements the predetermined lipid species measurements of ABI/ASI and the predetermined lipid species measurements of non-ABI/non-ASI (referred to as “control” or “normal”) to determine not only if the patient has ABI/ASI, but also the type of ABI/ASI (i.e. concussion, primary blast in blast-induced traumatic brain injury, electrical-induced brain injury (electrocution), seizure-induced injury, surgical-induced injury, stroke-induced injury, poison-induced injury, psychological injury, chemical injury, infectious injury, ischemic injury, metabolic injury, inflammatory injury, autoimmune injury, degenerative injury, hypoxic injury, and cancer/radiation-induced injury and so forth) and the prognosis.

The ACNSI libraries of predetermined lipid species measurements (ABI, ASI and controls) may be provided in a computer product (memory sticks, as an app for handheld devices such as tablets, pads, smart watches, cellular phones and so forth), or they may be uploaded to the memory of a computer system, including main frames, desktops, laptops, handheld devices such as tablets, pads, smart watches and cellular phones. Blood or any other bodily fluid, for example whole blood, blood plasma, blood serum, capillary blood sample, saliva, synovial fluid, urine, spinal fluid, bronchoalveolar lavage, tears, sweat, extracts, breath sample, volatile organic compounds (VOCs) and so forth, may be taken from a subject suspected of having an ABI and/or ASI. The VOCs are emitted from certain solids or liquids, for the latter, the VOCs may be measured from breath. Blood samples can be obtained using traditional blood draws or collected via dried plasma spots (DPS). In one embodiment, capillary blood is drawn from a puncture made on a finger using an incision device, such as a lancet or any other incision device [Lenicek Krleza J, Dorotic A, Grzunov A, Maradin M. Capillary blood sampling: National recommendations on behalf of the Croatian society of medical biochemistry and laboratory medicine. Biochem Medica. 2015; 25:335-58]. Blood is then applied to a blood collection device which is or contains a paper material (such as cellulose filter paper, a glass fibre membrane or other suitable materials). Blood may be applied to the device directly from the finger via a hanging drop or via a small pipette or capillary applicator. The device filters out red blood cells from the collected whole blood and permit plasma or serum to flow or soak into the paper collection material. This paper material is air dried and stored in an appropriate manner until extraction of the lipid species and analysis. Lipid species measurements may be obtained from the subject's sample using any known technology (for example, high performance liquid chromatography, thin layer chromatography, electrochemical analysis, mass spectrometry (MS), refractive index spectroscopy, ultra-violet spectroscopy, fluorescent analysis, radiochemical analysis, near-infrared spectroscopy, light scattering analysis, gas chromatography (GC), or GC coupled with MS, direct injection (DI) coupled with MS/MS, LC coupled with MS/MS and so forth). The subject's PC measurements may then be uploaded to the computer system (main frames, desktops, laptops, handheld devices such as phones, tablets, pads, watches, and so forth). An operator may then compare the subject's lipid species measurements with the predetermined set of lipid species measurements of ABI and/or ASI and the predetermined lipid species measurements of non-ABI/non-ASI (referred to as “control” or “normal”) to determine not only if the patient has ABI and/or ASI, but also the type of ABI and/or ASI, or whether a treatment is efficient.

Optimization of Biomarker Collection Via Dried Plasma Spot (DPS).

DPS can be easily sampled at home quickly, safely and less invasively than traditional blood draws, thereby allowing for repeat samples to be taken over the course of a season. However, the choice of plasma collection device is crucial to ensure a good quality, stable and quantitative sample of plasma that is easy and painless to procure. Reproducibility of sampling volume is determined by collecting replicate DPS, extracting GPLs, including PCs, with a suitable solvent, for example by a 4:1 methanol:chloroform solvent system, and comparing the precision of quantification of said GPLs for multiple DPS from various manufacturers. A coefficient of variation (CV) of repeated measurements of GPLs using a device must be <15%. Extraction efficiency and potential matrix effects from the devices is determined by comparing concentrations of neat standard solutions of GPLs to GPLs extracted from the DPS. Ideal recovery is 80-120% of each GPL, however, if extraction shows good reproducibility (i.e. <15% CV) it is acceptable. Stability of the compounds on the dried plasma cards is ascertained by collecting and storing cards under various conditions (i.e. room temperature, 4° C. and −20° C.) and quantifying GPLs over days, weeks and months to determine the extent of degradation of the analytes related to the specific filter paper in the collection device.

Optimization of Biomarker Measurement Using Mass Spectroscopy (MS).

Samples are analyzed by reversed phase liquid chromatography on a standard C18 column coupled to tandem MS/MS using a Waters TQ-S Micro MS. Quantitation is done using specific mass transitions pertaining to each individual GPL species and a stable isotope labelled GPL internal standard to maximize the analytical sensitivity and specificity of the MS. The device that maximizes GPL recovery and produces stable and reproducible results is used in all sampling going forward, and a full analytical validation is performed. This entails determining ‘accuracy’ (i.e. closeness of the measured GPL concentration to its true value should be within 15% of the true value), intra- and inter-day ‘precision’ (closeness of individual measurements to each other which should not deviate by more than 15%), ‘selectivity’ (ability to quantify the GPL of interest in the presence of other plasma metabolites), ‘stability’ of the analytes with respect to freeze-thaw cycles, short-term (<24 h), long-term (months) and post-preparative (in the LC instrument sample compartment prior to analysis), and a ‘calibration curve’ for limit of detection (lowest concentration detectable above noise), limit of quantification (lowest concentration that has a precision of ˜20%) and linear range of each GPL.

Returns to a normal level of lipid species may serve as an aid in following medical interventions (including rehabilitation therapy) of individuals affected by an ABI, ASI, mTSI, non-TSI, mTBI and/or non-TBI, and guide return to pre-ABI/pre-ASI play, school, work and/or daily activities.

As such, in another embodiment, the present disclosure is a method of tracking or following the efficiency of a medical intervention (including rehabilitation therapy) in an ACNSI patient, including mTSI patient, non-TSI patient, mTBI patient and non-TBI patient, the method including: (a) obtaining the levels of a lipid species or a cohort of (i.e. multiple) lipid species from the patient at different times during the medical intervention (including rehabilitation therapy); and (b) using univariate or multivariate statistical analysis and machine learning to compare the levels of the patient's single lipid species or cohort of lipid species during or at each of the different times with a predetermined set of metabolite profiles of ACNSI and a predetermined set of the single lipid species or cohort of lipid species of non-ACNSI (normal control) to follow the efficiency of the medical intervention in the patient. A return to a normal level of the single lipid species or cohort of lipid species of the patient may serve to assess whether the medical intervention (including rehabilitation therapy) of the patient has been successful.

In embodiments, the present disclosure is a method of assessing a non-human animal model of human ACNSI, including mTBI and non-TBI as well as mTSI and non-TSI. The method may be used for determining animal models that best represent the human condition, which may be useful for therapeutic intervention and discovery. The method, in one embodiment, may include: (a) obtaining the levels of a single lipid species or a cohort of lipid species from the non-human animal model of ACNSI; and (b) using univariate or multivariate statistical analysis and machine learning to compare the levels of the lipid species or cohort of lipid species in the non-human animal model with a predetermined set of the levels of the lipid species or cohort of lipid species of human ACNSI and a predetermined set of the levels of the single lipid species or cohort of lipid species of human non-ACNSI to determine if the non-human animal has ACNSI. The non-human animal model may be considered an accurate, reliable and reproducible model of human ACNSI if it is classified as ACNSI. The non-human animal model may be a model of human ACNSI if it is classified as ACNSI with a predetermined level of accuracy or certainty.

This disclosure also provides a kit for use in the methods described herein containing one or more reagents for use in the detection of the GPLs, or a cohort of PCs, in a biological sample according to the methods of the present disclosure, and instructions for use. The instructions may also include instructions to treat the subject upon a diagnosis of ACNSI.

In order to aid in the understanding and preparation of the within disclosure, the following illustrative, non-limiting, examples are provided.

### EXAMPLES

### Example 1

In this Example, we report the measurements of 71 individual blood lipid species following adolescent concussion, to which 26 lipid species were significantly decreased after sports related concussion (P<0.01). We also report a simplified diagnostic model utilizing the leading 4 GPLs with maximal change and greatest AUCs on ROC curve analyses. In addition, we demonstrate partial PC return to baseline during clinical recovery.

AIM: to determine if plasma lipid species alone could aid concussion diagnosis as well as recovery.

This study was approved by Western University Human Ethics Review Board.

Subjects:

Male adolescent ice hockey athletes (Bantam Division; aged 12-14 years) were recruited to participate in this study. Patients suspected to have suffered a concussion were clinically evaluated at our academic Sports Medicine Clinic within 72 h of the injury, a time frame to account for weekend injuries. They were either referred by other healthcare providers, including emergency physicians, family physicians, coaches, and/or trainers, or they had booked an appointment by self-referral. Concussion was diagnosed when there was an observed mechanism of injury followed by onset of typical concussive symptoms, and the absence of structural injury (i.e., no focal neurological abnormalities on examination). Control athletes consisted of non-injured hockey players that were age- and sex-matched, and that had not suffered a concussion in the past 6 months. Any subject with a reported neurological disease was excluded. Concussed and control athletes, including their parents/guardians, completed a Sport Concussion Assessment Tool-3rd edition [SCAT3 (18); 13-14 years of age] or a Child-SCAT3 (19) (a modified tool recommended for children 12 years of age or younger that considers developmental differences in performance)], as well as a complete history, physical and neurologic examination by a Primary Care Sport Medicine Physician with expertise in concussion management. All injured athletes were provided with standardized concussion care.

Blood Collection and Analyses:

All athletes on the first clinic visit had 20 ml of blood drawn into EDTA Vacutainer tubes. The blood was centrifuged, the plasma aliquoted into cryovials at a volume of 500 μl and stored at −80° C. Freeze/thaw cycles were avoided. Plasma was collected by strict standard operating procedures, with equal processing times between cohorts. No restrictions were placed on any subjects (e.g., fasting), and thus, they were considered to be in their natural state.

A targeted quantitative metabolomics approach was applied to analyze the plasma samples using a combination of direct injection mass spectrometry (AbsoluteIDQ™ Kit) with a reverse-phase LC/MS/MS Kit (BIOCRATES Life Sciences AG, Austria), as previously described (15). The method combines the derivatization and extraction of analytes, and selective mass spectrometric detection using multiple reaction monitoring pairs (standards are integrated in the Kit plate filter for metabolite quantification). Briefly, plasma samples were thawed on ice and then vortexed and centrifuged at 13,000 g. Each plasma sample (10 μl) was loaded onto the center of the filter on the upper 96-well kit plate and dried in a stream of nitrogen. Subsequently, 20 μl of a 5% solution of phenyl-isothiocyanate was added for derivatization. After incubation, the filter spots were dried again using an evaporator. Extraction of the metabolites was then achieved by adding 300 μl methanol containing 5 mM ammonium acetate. The extracts were obtained by centrifugation into the lower 96-deep well plate, followed by a dilution step with kit MS running solvent. Mass spectrometric analysis was performed on an API4000 Qtrap® tandem mass spectrometry instrument (Applied Biosystems/MDS Analytical Technologies, Foster City, Calif.) equipped with a solvent delivery system. The samples were delivered to the LC mass spectrometer by DI. The Biocrates MetIQ software was used to control the entire assay workflow, from sample registration to automated calculation of metabolite concentrations. A targeted profiling scheme was used to quantitatively screen for known small molecule metabolites using multiple reaction monitoring, neutral loss and precursor ion scans.

A total of 71 lipid species were quantitatively measured: PC aa (C24:0, C28:1, C30:0, C30:2, C32:0, C32:1, C32:2, C32:3, C34:1, C34:2, C34:3, C34:4, C36:0, C36:1, C36:2, C36:3, C36:4, C36:5, C36:6, C38:0, C38:1, C38:3, C38:4, C38:5, C38:6, C40:2, C40:3, C40:4, C40:5, C40:6, C42:0, C42:1, C42:4, C42:5, C42:6), and PC ae (C30:0, C30:1, C32:1, C32:2, C34:0, C34:1, C34:2, C34:3, C36:0, C36:1, C36:2, C36:3, C36:4, C36:5, C38:0, C38:1, C38:2, C38:3, C38:4, C38:5, C38:6 C40:1, C40:2, C40:3, C40:4, C40:5, C40:6, C42:1, C42:2, C42:3, C42:4, C42:5, C44:3, C44:4, C44:5, C44:6).

Statistical Analyses:

Demographic, concussion tool data and lipid species concentrations were reported as mean±standard error (SE). Given the number of lipid species analyzed and the risk of false positives, a P value <0.01 was used as our standard of statistical significance. Receiver operating characteristic (ROC) curves were conducted to determine sensitivity and specificity of individual PCs for predicting concussion. Area-under-the-curve (AUC) was calculated for each lipid species, with an AUC greater than 0.7 considered as acceptable (20). The coordinates of the curves were then analyzed to identify cut-off values based on the highest sensitivity and specificity for predicting concussion. Logistic regression analyses were also conducted with concussion as the outcome and the top four lipid species with AUC>0.85 entered as predictors; the predicted values from the logistic regression models were then saved for use in ROC curve analyses to determine the most parsimonious combination of lipid species with the greatest combined AUC. All analyses were conducted using SPSS version 25 (IBM Corp., Armonk, N.Y., USA).

Results

We investigated a total 12 concussed athletes (13.4±2.3 years of age) and 17 age- and sex-matched controls (12.9±1.0 years of age; P=0.213). The estimated time from concussion occurrence to blood draw at the first clinic visit was 2.3±0.7 days. Headache was the most prevalent self-reported symptom, occurring in 91% of concussion patients. Self-reported symptom evaluation as per SCAT3 (n=11) revealed a total symptom score and a total symptom severity of 11.6±4.8 and 29.3±22.8, respectively. In contrast, the non-concussed athletes had a total symptom score and a total symptom severity of 0.5±1.5 and 0.6±1.8, respectively. One concussed athlete was evaluated with the Child SCAT; total symptom score of 6 and a total symptom severity of 12 (the parent score indicated a total symptom score of 3 and a total symptom severity of 8). Collectively, these self-reported symptoms and the symptom severity scores indicated that the athletes suffered a mild-moderate traumatic brain injury.

71 lipid species were analyzed for statistically significant changes within 72 hours of injury and determined their individual receiver operating characteristic (ROC) curves.

The data obtained demonstrates that 53 of the 71 measured lipid species were significantly decreased after sports related concussion (p<0.05). 26 lipid species had a statistically significant decrease in concentration after concussion (P<0.01; Table 1). None of the lipid species measured increased after concussion.

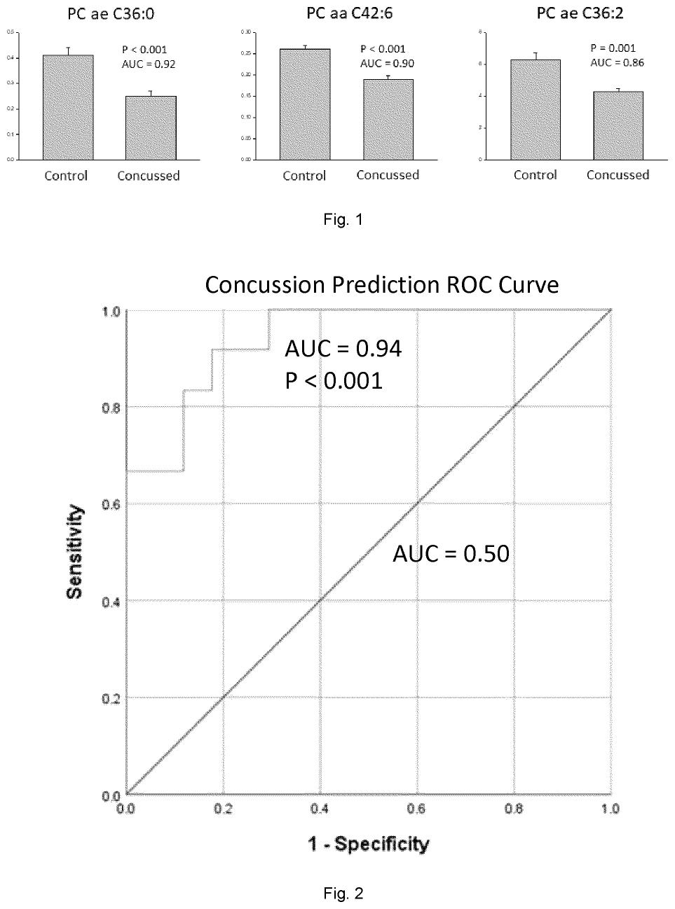

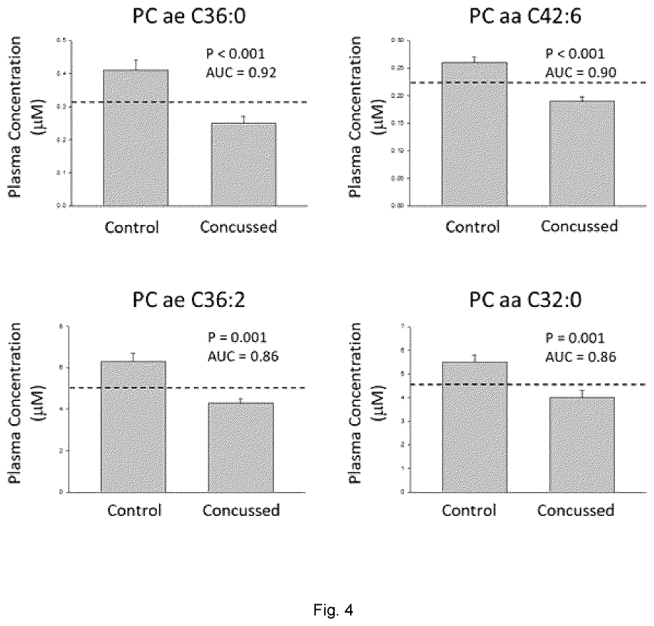

ROC curve analyses identified that 3 (FIG. 1), or 4 (FIG. 4) glycerophospholipids (GPLs) had AUCs of 0.86 or greater for concussion diagnosis.

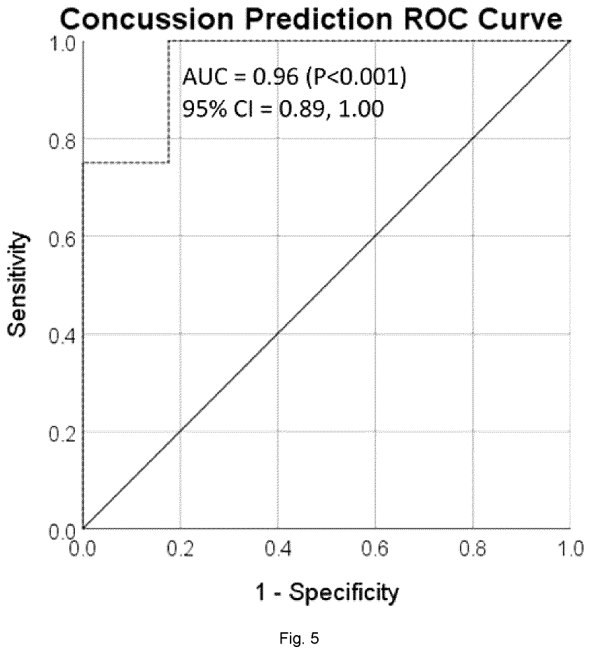

Importantly, combining these cohorts of 3 and 4 GPLs produced an AUC of 0.94 (FIG. 2) and 0.96 (FIG. 5) respectively for concussion diagnosis. The top 3 GPLs and top 4 GPLs with a combination of the lowest P-value and highest AUC are shown in FIG. 1 (PCaeC36:0, PCaaC42:6, and PCaeC36:2; P<0.001) and 4 (PCaeC36:0, PCaaC42:6, PCaeC36:2 and PCaaC32:0; P≤0.001). In the case of FIG. 4, cut off values in μM were ≤ to 0.31 for PCaeC36:0, 0.22 for PCaaC42:6, 5.07 for PCaeC36:2 and 4.63 for PCaaC32:0. When the predicted values for the 4 GPLs, as determined by regression analyses, were combined, the AUC increased to 0.96 (FIG. 5; P<0.001). The addition or subtraction of other lipid species failed to improve the model.

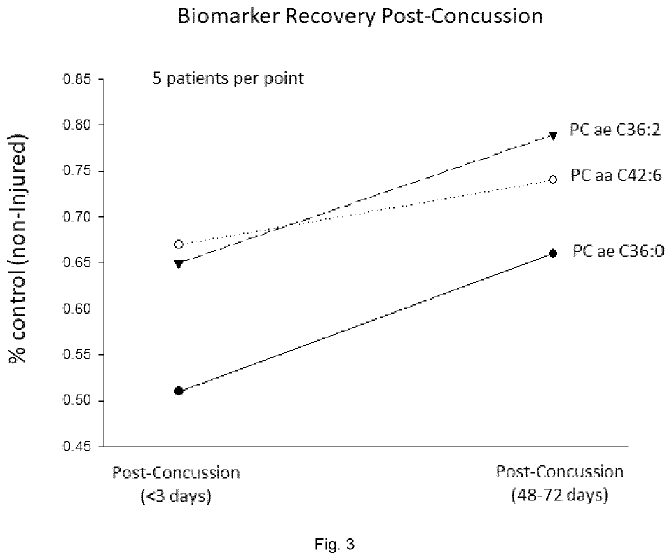

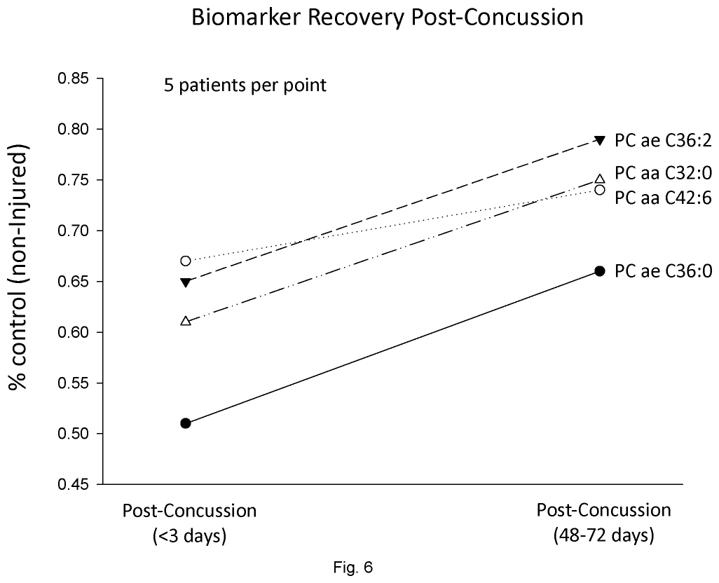

A subset of concussed athletes consented to a further blood draw during recovery (n=5; FIGS. 3 and 6). Each of the 3 (FIG. 3) or 4 (FIG. 6) GPLs recovered, albeit partially, indicating that biochemical recovery occurred in parallel with concussion symptom resolution. The identified 3 and 4 GPLs all demonstrated biochemical recovery that was temporally associated with medical clearance (FIGS. 3 and 6).

The data presented herein indicates that cohorts of lipid species may provide excellent diagnosis for concussion. Recovery from concussion was reflected in plasma PC level recovery.

In this study, we show that a large number of blood lipid species are decreased after concussion, with 4 individual GPLs having very good to excellent diagnostic potential (AUC>0.85). Combining the 4 GPLs resulted in a potentially excellent diagnostic test with an AUC of 0.96. Furthermore, partial recovery of the 4 GPLs paralleled clinical symptom resolution, indicating that the combination of the 4 GPLs could also aid clinical care as a potential recovery biomarker.

Concussion diagnosis remains problematic. Patients often have difficulty recognizing injury induced deficits and in quantifying their symptoms. Concussion diagnostics are further complicated by patient and injury heterogeneity. Indeed, every athlete and injury are different. Finally, injury symptoms may be underreported, or denied altogether, for secondary gain [i.e. an athlete that wants to continue play; (21, 22)]. Currently, concussion diagnosis relies on clinical criteria alone, but concussion diagnostics tools are imperfect. The SCAT is at best 89% accurate (9), with certain reported symptoms unhelpful in the adolescent population, including sleep disturbances, anxiety, irritability, emotional level and sadness (9, 10). Thus, an accurate and easy to use biomarker would be invaluable for concussion diagnostics.

Once a concussion has occurred, a graded return to activities with symptom resolution is recommended (4). Medical clearance requires resolution in self-reported symptoms and clinical signs. If return to sport occurs prematurely for any reason, the athlete is at increased risk of injury, with potentially serious consequences, including second impact syndrome (23). Our data suggests that brain injury induced biochemical changes may not have fully resolved with self-reported symptoms and clinical signs. Indeed, our previous MR imaging of these same adolescent athletes 3 months post-injury suggested that there were diffusion abnormalities within multiple white matter tracts and functional hyper-connectivity (24). Importantly, MR spectroscopy demonstrated persistently decreased choline, consistent with our finding that the 4 choline-containing GPLs measured here were still depressed at medical clearance.

The consistent depression in blood lipid species concentrations measured with mass spectrometry is novel and intriguing, but the underlying pathophysiology unknown. With concussion, one can only speculate that depressed lipid species may indicate post-injury demand for membrane repair or alternatively, less cellular activity and/or turnover. Of the 4 GPLs utilized in our prediction model, 2 GPLs were plasmalogens that are present in significant amounts in myelin and may play an essential role as an antioxidant due to their vinyl ether bond, which has a high reactivity with oxygen (25).

While we demonstrated a reduction in blood lipid species after concussion, other brain injury biomarkers increase after concussion (14). The protein brain injury biomarkers, as measured with antibody techniques, are released after injury and are relatively specific to a wide variety of brain cells, including neurons (UCH-L1, NF-L, Tau, NSE, SNTF), astrocytes (GFAP, S100 beta and oligodendrocytes (MBP). To date, only a handful of brain injury protein biomarkers have shown some degree of diagnostic accuracy, such as a combination of GFAP and UCH-L1 yielding an AUC 0.71 (26).

Our data raised an additional question relating to potential therapeutics: Could dietary supplementation of lipid species improve or hasten concussion recovery? In both animal and human studies, lipid species supplementation has been demonstrated to slow cerebral structure decline with age and support cognitive functioning (27). Additional mechanisms that lipid species influence brain health include decreased reactive oxygen species, depressed pro-inflammatory cytokines and reduced homocysteine.

Our study evaluated only a small number of adolescent athletes. However, to identify such a strong predictive model with high statistical significance illustrates the potential of lipid species for diagnostic utility. With regards to recovery, many athletes refused repeat venipuncture limiting our numbers, however a clear trend in biochemical recovery was uncovered. Second, we did not have baseline measurements from each athlete and therefore, we compared concussed athletes to a control cohort. However, our control cohort was optimized as they consisted of age-, sex- and activity-matched athletes. Third, we are unclear of the anatomical origin of the lipid species changes, however, given that lipid species are highly enriched in brain and that MR spectroscopy on these same injured athletes demonstrated reduced brain choline, a major constituent of lipid species, we suggest that the lipid species changes have a brain origin. Ultimately, future studies should endeavour to have a larger cohort of athletes with measurements at baseline, post-injury and multiple intervals during recovery.

In summary, we show that a single lipid species or a combination of lipid species accurately diagnoses concussion in adolescent athletes. Lipid species are not only acutely depressed post-injury, but also recover with clinical improvement, albeit not completely. These data suggest that decreased plasma lipid species are novel biomarkers of mild traumatic brain injury and its recovery.

TABLE 1

| | Concussed (n = 12) | | Controls (n = 17) | | | |
|---|---|---|---|---|---|---|
| Lipid Species | Mean (SE) | | Mean (SE) | | P-value | AUC |
| PC aa C24:0 | 0.07 | (0.02) | 0.07 | (0.01) | 0.696 | 0.61 |
| PC aa C28:1 | 0.77 | (0.10) | 0.91 | (0.06) | 0.193 | 0.72 |
| PC aa C30:0 | 0.82 | (0.06) | 1.10 | (0.08) | 0.014 | 0.77 |
| PC aa C30:2 | 0.10 | (0.01) | 0.13 | (0.02) | 0.153 | 0.66 |
| PC aa C32:0 | 3.98 | (0.26) | 5.55 | (0.33) | 0.002* | 0.86 |
| PC aa C32:1 | 3.44 | (0.44) | 5.21 | (0.47) | 0.014 | 0.75 |
| PC aa C32:2 | 1.02 | (0.11) | 1.54 | (0.13) | 0.009* | 0.79 |
| PC aa C32:3 | 0.17 | (0.11) | 0.24 | (0.01) | 0.001* | 0.84 |
| PC aa C34:1 | 62.21 | (3.64) | 81.69 | (5.14) | 0.008* | 0.77 |
| PC aa C34:2 | 141.09 | (6.30) | 191.51 | (11.50) | 0.001* | 0.84 |
| PC aa C34:3 | 4.93 | (0.38) | 7.18 | (0.57) | 0.006* | 0.80 |
| PC aa C34:4 | 0.50 | (0.07) | 0.70 | (0.05) | 0.024 | 0.73 |
| PC aa C36:0 | 0.58 | (0.06) | 0.91 | (0.15) | 0.090 | 0.74 |
| PC aa C36:1 | 15.07 | (1.18) | 19.82 | (1.23) | 0.012 | 0.79 |
| PC aa C36:2 | 86.94 | (5.40) | 115.89 | (7.01) | 0.005* | 0.81 |
| PC aa C36:3 | 45.14 | (2.29) | 58.25 | (4.89) | 0.024 | 0.74 |
| PC aa C36:4 | 58.46 | (4.97) | 75.12 | (5.11) | 0.032 | 0.74 |
| PC aa C36:5 | 4.40 | (0.48) | 8.07 | (1.46) | 0.057 | 0.80 |
| PC aa C36:6 | 0.19 | (0.02) | 0.31 | (0.03) | 0.007* | 0.80 |
| PC aa C38:0 | 0.75 | (0.08) | 1.04 | (0.16) | 0.170 | 0.71 |
| PC aa C38:1 | 0.27 | (0.04) | 0.38 | (0.06) | 0.231 | 0.60 |
| PC aa C38:3 | 15.44 | (1.12) | 19.47 | (1.91) | 0.114 | 0.68 |
| PC aa C38:4 | 36.17 | (3.29) | 42.48 | (3.02) | 0.175 | 0.65 |
| PC aa C38:5 | 16.71 | (1.30) | 22.25 | (1.71) | 0.016 | 0.74 |
| PC aa C38:6 | 18.72 | (1.70) | 27.33 | (4.49) | 0.133 | 0.69 |
| PC aa C40:2 | 0.09 | (0.01) | 0.12 | (0.01) | 0.006* | 0.77 |
| PC aa C40:3 | 0.15 | (0.01) | 0.18 | (0.02) | 0.201 | 0.56 |
| PC aa C40:4 | 1.15 | (0.07) | 1.44 | (0.11) | 0.058 | 0.70 |
| PC aa C40:5 | 3.16 | (0.26) | 4.05 | (0.33) | 0.059 | 0.71 |
| PC aa C40:6 | 6.33 | (0.67) | 9.09 | (1.65) | 0.190 | 0.70 |
| PC aa C42:0 | 0.17 | (0.02) | 0.19 | (0.02) | 0.242 | 0.64 |
| PC aa C42:1 | 0.08 | (0.01) | 0.10 | (0.01) | 0.202 | 0.63 |
| PC aa C42:4 | 0.06 | (0.00) | 0.07 | (0.00) | 0.043 | 0.73 |
| PC aa C42:5 | 0.11 | (0.01) | 0.13 | (0.01) | 0.096 | 0.63 |
| PC aa C42:6 | 0.19 | (0.01) | 0.26 | (0.01) | <0.001* | 0.90 |
| PC ae C30:0 | 0.13 | (0.01) | 0.17 | (0.01) | 0.001* | 0.83 |
| PC ae C30:1 | 0.06 | (0.01) | 0.08 | (0.01) | 0.107 | 0.78 |
| PC ae C32:1 | 0.80 | (0.06) | 1.09 | (0.06) | 0.004* | 0.80 |
| PC ae C32:2 | 0.20 | (0.02) | 0.28 | (0.02) | 0.004* | 0.80 |
| PC ae C34:0 | 0.39 | (0.04) | 0.59 | (0.04) | 0.001* | 0.85 |
| PC ae C34:1 | 2.92 | (0.15) | 3.91 | (0.23) | 0.003* | 0.86 |
| PC ae C34:2 | 3.40 | (0.21) | 4.76 | (0.28) | 0.001* | 0.85 |
| PC ae C34:3 | 2.67 | (0.20) | 4.01 | (0.27) | 0.001* | 0.85 |
| PC ae C36:0 | 0.25 | (0.02) | 0.41 | (0.03) | <0.001* | 0.92 |
| PC ae C36:1 | 2.22 | (0.14) | 3.36 | (0.27) | 0.001* | 0.85 |
| PC ae C36:2 | 4.33 | (0.24) | 6.32 | (0.44) | 0.001* | 0.86 |
| PC ae C36:3 | 2.46 | (0.15) | 3.39 | (0.21) | 0.002* | 0.83 |
| PC ae C36:4 | 5.77 | (0.56) | 7.21 | (0.36) | 0.031 | 0.76 |
| PC ae C36:5 | 3.82 | (0.42) | 4.74 | (0.31) | 0.079 | 0.71 |
| PC ae C38:0 | 0.55 | (0.04) | 0.77 | (0.07) | 0.028 | 0.78 |
| PC ae C38:1 | 0.24 | (0.03) | 0.43 | (0.06) | 0.006* | 0.75 |
| PC ae C38:2 | 0.69 | (0.05) | 1.05 | (0.09) | 0.002* | 0.83 |
| PC ae C38:3 | 1.23 | (0.07) | 1.81 | (0.15) | 0.002* | 0.83 |
| PC ae C38:4 | 4.38 | (0.31) | 5.60 | (0.32) | 0.013 | 0.77 |
| PC ae C38:5 | 6.40 | (0.57) | 7.32 | (0.35) | 0.160 | 0.68 |
| PC ae C38:6 | 2.11 | (0.22) | 2.86 | (0.31) | 0.084 | 0.73 |
| PC ae C40:1 | 0.34 | (0.04) | 0.42 | (0.02) | 0.075 | 0.69 |
| PC ae C40:2 | 0.38 | (0.02) | 0.50 | (0.03) | 0.004* | 0.79 |
| PC ae C40:3 | 0.30 | (0.02) | 0.42 | (0.03) | 0.002* | 0.83 |
| PC ae C40:4 | 0.74 | (0.04) | 0.87 | (0.04) | 0.048 | 0.71 |
| PC ae C40:5 | 1.10 | (0.07) | 1.34 | (0.06) | 0.016 | 0.78 |
| PC ae C40:6 | 1.19 | (0.09) | 1.69 | (0.20) | 0.051 | 0.77 |
| PC ae C42:1 | 0.14 | (0.01) | 0.17 | (0.01) | 0.064 | 0.72 |
| PC ae C42:2 | 0.14 | (0.01) | 0.17 | (0.01) | 0.047 | 0.72 |
| PC ae C42:3 | 0.19 | (0.01) | 0.22 | (0.01) | 0.112 | 0.68 |
| PC ae C42:4 | 0.24 | (0.02) | 0.28 | (0.02) | 0.177 | 0.74 |
| PC ae C42:5 | 0.84 | (0.04) | 0.86 | (0.03) | 0.647 | 0.53 |
| PC ae C44:3 | 0.04 | (0.00) | 0.04 | (0.00) | 0.142 | 0.70 |
| PC ae C44:4 | 0.13 | (0.08) | 0.14 | (0.01) | 0.308 | 0.63 |
| PC ae C44:5 | 0.58 | (0.05) | 0.59 | (0.04) | 0.888 | 0.49 |
| PC ae C44:6 | 0.41 | (0.04) | 0.43 | (0.03) | 0.659 | 0.58 |

### REFERENCES

1. http://www.cdc.gov/TraumaticBrainInjury.
2. Bakhos L L, Lockhart G R, Myers R, Linakis J G. Emergency department visits for concussion in young child athletes. Pediatrics. 2010; 126(3):e550-6.
3. Stewart T C, Gilliland J, Fraser D D. An epidemiologic profile of pediatric concussions: identifying urban and rural differences. The journal of trauma and acute care surgery. 2014; 76(3):736-42.
4. McCrory P, Meeuwisse W, Aubry M, Cantu B, Dvorak J, Echemendia R J, et al. Consensus statement on concussion in sport—the 4th International Conference on Concussion in Sport held in Zurich, November 2012. Clin J Sport Med. 2013; 23(2):89-117.
5. Asken B M, Snyder A R, Smith M S, Zaremski J L, Bauer R M. Concussion-like symptom reporting in non-concussed adolescent athletes. Clin Neuropsychol. 2017; 31(1):138-53.
6. Smith-Seemiller L, Fow N R, Kant R, Franzen M D. Presence of post-concussion syndrome symptoms in patients with chronic pain vs mild traumatic brain injury. Brain Inj. 2003; 17(3):199-206.
7. Karlin A M. Concussion in the pediatric and adolescent population: “different population, different concerns”. P M R. 2011; 3(10 Suppl 2):5369-79.
8. Patel D R, Reddy V. Sport-related concussion in adolescents. Pediatr Clin North Am. 2010; 57(3):649-70.
9. Babl F E, Dionisio D, Davenport L, Baylis A, Hearps SJC, Bressan S, et al. Accuracy of Components of SCAT to Identify Children With Concussion. Pediatrics. 2017; 140(2).
10. Harriss A B, Abbott K C, Humphreys D, Daley M, Moir M E, Woehrle E, et al. Concussion Symptoms Predictive of Adolescent Sport-Related Concussion Injury. Clin J Sport Med. 2019.
11. Barlow K M, Crawford S, Stevenson A, Sandhu S S, Belanger F, Dewey D. Epidemiology of postconcussion syndrome in pediatric mild traumatic brain injury. Pediatrics. 2010; 126(2):e374-81.
12. Field M, Collins M W, Lovell M R, Maroon J. Does age play a role in recovery from sports-related concussion? A comparison of high school and collegiate athletes. J Pediatr. 2003; 142(5):546-53.
13. Taylor H G, Dietrich A, Nuss K, Wright M, Rusin J, Bangert B, et al. Post-concussive symptoms in children with mild traumatic brain injury. Neuropsychology. 2010; 24(2):148-59.
14. Kim H J, Tsao J W, Stanfill A G. The current state of biomarkers of mild traumatic brain injury. JCI Insight. 2018; 3(1).
15. Daley M, Dekaban G, Bartha R, Brown A, Charyk Stewart T, Doherty T, et al. Metabolomics profiling of concussion in adolescent male hockey players: a novel diagnostic method. Metabolomics.
16. O'Brien J S, Sampson E L. Lipid composition of the normal human brain: gray matter, white matter, and myelin. J Lipid Res. 1965; 6(4):537-44.
17. Farooqui A A, Horrocks L A, Farooqui T. Glycerophospholipids in brain: their metabolism, incorporation into membranes, functions, and involvement in neurological disorders. Chemistry and physics of lipids. 2000; 106(1):1-29.
18. Guskiewicz K M, Register-Mihalik J, McCrory P, McCrea M, Johnston K, Makdissi M, et al. Evidence-based approach to revising the SCAT2: introducing the SCAT3. Br J Sports Med. 2013; 47(5):289-93.
19. Glaviano N R, Benson S, Goodkin H P, Broshek D K, Saliba S. Baseline SCAT2 Assessment of Healthy Youth Student-Athletes: Preliminary Evidence for the Use of the Child-SCAT3 in Children Younger Than 13 Years. Clin J Sport Med. 2015; 25(4):373-9.
20. Zhu W, Zeng N, Wang N. Sensitivity, Specificity, Accuracy, Associated Confidence Interval and ROC Analysis with Practical SAS® Implementations. Northeast SAS Users Group Proceedings. 2010; http://www.lexiansen.com/nesug/nesug10/hl/hl07.pdf.
21. Cusimano M D, Topolovec-Vranic J, Zhang S, Mullen S J, Wong M, Ilie G. Factors Influencing the Underreporting of Concussion in Sports: A Qualitative Study of Minor Hockey Participants. Clin J Sport

Med. 2017; 27(4):375-80.
22. Meier T B, Brummel at Singh R, Nerio O, Polanski D W, Bellgowan P S. The underreporting of self-reported symptoms following sports-related concussion. J Sci Med Sport. 2015; 18(5):507-11.
23. McLendon L A, Kralik S F, Grayson P A, Golomb M R. The Controversial Second Impact Syndrome: A Review of the Literature. Pediatr Neurol. 2016; 62:9-17.
24. Manning K Y, Schranz A, Bartha R, Dekaban G A, Barreira C, Brown A, et al. Multiparametric MRI changes persist beyond recovery in concussed adolescent hockey players. Neurology. 2017; 89(21):2157-66.
25. Braverman N E, Moser A B. Functions of plasmalogen lipids in health and disease. Biochim Biophys Acta. 2012; 1822(9):1442-52.
26. McCrea M, Broglio S P, McAllister T W, Gill J, Giza C C, Huber D L, et al. Association of Blood Biomarkers With Acute Sport-Related Concussion in Collegiate Athletes: Findings From the NCAA and Department of Defense CARE Consortium. JAMA Netw Open. 2020; 3(1):e1919771.
27. Reddan J M, White D J, Macpherson H, Scholey A, Pipingas A. Glycerophospholipid Supplementation as a Potential Intervention for Supporting Cerebral Structure in Older Adults. Front Aging Neurosci. 2018; 10:49.

Through the embodiments that are illustrated and described, the currently contemplated best mode of making and using the disclosure is described. Without further elaboration, it is believed that one of ordinary skill in the art can, based on the description presented herein, utilize the present disclosure to the full extent. All publications cited herein are incorporated by reference.

Although the description above contains many specificities, these should not be construed as limiting the scope of the disclosure, but as merely providing illustrations of some of the presently embodiments of this disclosure.

1. A method of diagnosing mild traumatic brain injury (mTBI) in a subject comprising:
(a) obtaining a test sample from the subject, and
(b) comparing levels of a lipid species or a cohort of multiple lipid species in the test sample with the levels of the lipid species or the cohort of multiple lipid species in a reference normal control sample using quantitative measurements, wherein a decrease in the level of the lipid species or the cohort of multiple lipid species in the test sample relative to the reference normal control sample is indicative of mTBI in the subject, and
(c) treating the subject for mTBI when there is a decrease in the level of the lipid species or the cohort of multiple lipid species.

2. The method of claim 1, wherein the method further comprises obtaining a sample from the subject during the subject's recovery for mTBI, wherein an increase in the levels of the lipid species or the cohort of multiple lipid species in the recovery sample relative to the levels obtained in the test sample is indicative of a normalization of the subject.

3. (canceled)

4. (canceled)

5. The method of claim 1, wherein the reference normal control sample is obtained from the subject at baseline.

6. (canceled)

7. The method of claim 1, wherein the mTBI is concussion or blast wave injury.

8. (canceled)

9. The method of claim 1, wherein the mTBI is concussion, and wherein step (b) comprises comparing the levels of the cohort of multiple lipid species, the cohort of multiple lipid species including PC ae C36:0, PC aa C42:6 and PC ae C36:2.

10. The method of claim 1, wherein the mTBI is concussion, and wherein step (b) comprises comparing the levels of the cohort of multiple lipid species, the cohort of multiple lipid species including PC ae C36:0, PC aa C42:6, PC ae C36:2 and PC aa C32:0.

11. The method of claim 1, wherein the lipid species or the cohort of multiple lipid species include PC aa C30:0, PC aa C32:0, PC aa C32:1, PC aa C32:2, PC aa C32:3, PC aa C34:1, PC aa C34:2, PC aa C34:3, PC aa C34:4, PC aa C36:1, PC aa C36:2, PC aa C36:3, PC aa C36:4, PC aa C36:6, PC aa C38:5, PC aa C40:2, PC aa C42:4, PC aa C42:5, PC aa C42:6, PC ae C30:0, PC ae C32:1, PC ae C32:2, PC ae C34:0, PC ae C34:1, PC ae C34:2, PC ae C34:3, PC ae C36:0, PC ae C36:1, PC ae C36:2, PC ae C36:3, PC ae C36:4, PC ae C38:0, PC ae C38:1, PC ae C38:2, PC ae C38:3, PC ae C38:4, PC ae C40:2, PC ae C40:3, PC ae C40:4, PC ae C40:5, and PC ae C42:2.

12. The method of claim 1, wherein the mTBI ACNSI is concussion, and wherein step (b) comprises comparing the levels of the lipid species, and wherein the lipid species is at least one of PC ae C36:0, PC aa C42:6, PC ae C36:2 or PC aa C32:0.

13. The method of claim 1, wherein the sample is a blood sample, a plasma sample, a serum sample, a capillary sample, a sweat sample, a tear sample, a breath sample or a combination thereof.

14-21. (canceled)

22. A mild traumatic brain injury (mTBI) diagnostic apparatus, the mTBI diagnostic apparatus including a computer readable storage medium and a computer program mechanism embedded therein, the computer program mechanism comprising executable instructions for performing a method of diagnosing mTBI in a subject, said executable instructions comprising: (a) comparing levels of a lipid species or a cohort of multiple lipid species in a test sample of the subject obtained after an injury of the central nervous system, with the levels of the lipid species or the cohort of multiple lipid species in a reference normal control sample, and (b) providing an mTBI positive signal to seek mTBI treatment when there is a decrease in the level of the lipid species or in the levels of the cohort of multiple lipid species in the test sample relative to the reference normal control sample is indicative of mTBI.

23. The mTBI diagnostic apparatus of claim 22, wherein the instructions further include comparing the levels of the lipid species or of the levels of the cohort of multiple lipid species of the subject, with the levels of the lipid species or of the cohort of multiple lipid species in a sample obtained from the subject during the subject's treatment of mTBI, wherein an increase in the level of the lipid species or in the levels of the cohort of multiple lipid species during the treatment relative to the levels of the lipid species or the cohort of multiple lipid species in the test ample is indicative of a normalization of the subject.

24. (canceled)

25. (canceled)

26. The mTBI diagnostic apparatus of claim 22, wherein the reference normal control sample is obtained from the subject at baseline.

27. (canceled)

28. The mTBI diagnostic apparatus of claim 22, wherein the mTBI is concussion or blast wave injury.

29. The mTBI diagnostic apparatus of claim 22, wherein the mTBI is concussion and instruction (a) comprises comparing the levels of the cohort of multiple lipid species, the cohort of multiple lipid species including PC ae C36:0, PC aa C42:6 and PC ae C36:2.

30. The mTBI diagnostic apparatus of claim 22, wherein the mTBI is concussion, and instruction (a) comprises comparing the levels of the cohort of multiple lipid species, the cohort of multiple lipid species including PC ae C36:0, PC aa C42:6, PC ae C36:2 and PC aa C32:0.

31. The mTBI diagnostic apparatus of claim 22, wherein the mTBI is concussion and wherein the lipid species is at least one of PC ae C36:0, PC aa C42:6, PC ae C36:2 and PC aa C32:0.

32. The mTBI diagnostic apparatus of claim 22, wherein the lipid species or cohort of multiple lipid species include PC aa C30:0, PC aa C32:0, PC aa C32:1, PC aa C32:2, PC aa C32:3, PC aa C34:1, PC aa C34:2, PC aa C34:3, PC aa C34:4, PC aa C36:1, PC aa C36:2, PC aa C36:3, PC aa C36:4, PC aa C36:6, PC aa C38:5, PC aa C40:2, PC aa C42:4, PC aa C42:5, PC aa C42:6, PC ae C30:0, PC ae C32:1, PC ae C32:2, PC ae C34:0, PC ae C34:1, PC ae C34:2, PC ae C34:3, PC ae C36:0, PC ae C36:1, PC ae C36:2, PC ae C36:3, PC ae C36:4, PC ae C38:0, PC ae C38:1, PC ae C38:2, PC ae C38:3, PC ae C38:4, PC ae C40:2, PC ae C40:3, PC ae C40:4, PC ae C40:5, and PC ae C42:2.

33-44. (canceled)

45. The method of claim 1, wherein the treating comprises increasing in the subject the level of the lipid species or of the cohort of multiple lipid species.

46. The method of claim 1, wherein the treating comprises administering to the subject symptom therapies and/or rehabilitation steps.

47. The method of claim 1, wherein the lipid species or the cohort of multiple lipid species include PC aa C32:0, PC aa C32:2, PC aa C32:3, PC aa C34:1, PC aa C34:2, PC aa C34:3, PC aa C36:2, PC aa C36:6, PC aa C40:2, PC aa C42:6, PC ae C30:0, PC ae C32:1, PC ae C32:2, PC ae C34:0, PC ae C34:1, PC ae C34:2, PC ae C34:3, PC ae C36:0, PC ae C36:1, PC ae C36:2, PC ae C36:3, PC ae C38:1, PC ae C38:2, PC ae C38:3, PC ae C40:2 and PC ae C40:3.

48. The method of claim 1, wherein the lipid species or the cohort of multiple lipid species is a phosphatidylcholine.

49. The mTBI diagnostic apparatus of claim 22, wherein the lipid species or the cohort of multiple lipid species include PC aa C32:0, PC aa C32:2, PC aa C32:3, PC aa C34:1, PC aa C34:2, PC aa C34:3, PC aa C36:2, PC aa C36:6, PC aa C40:2, PC aa C42:6, PC ae C30:0, PC ae C32:1, PC ae C32:2, PC ae C34:0, PC ae C34:1, PC ae C34:2, PC ae C34:3, PC ae C36:0, PC ae C36:1, PC ae C36:2, PC ae C36:3, PC ae C38:1, PC ae C38:2, PC ae C38:3, PC ae C40:2 and PC ae C40:3.

50. The mTBI diagnostic apparatus of claim 22, wherein the lipid species or the cohort of multiple lipid species is a phosphatidylcholine.
